(19) United States
(12) Patent Application Publication
(10) Pub. No.: US 2017/0023475 A1
(43) Pub. Date: Jan. 26, 2017

(54) SINGLE ELEMENT HYDROGEN SENSING MATERIAL BASED ON HAFNIUM

(71) Applicant: Technische Universiteit Delft, Delft (NL)

(72) Inventors: Bernard Dam, Delft (NL); Christiaan Boelsma, Delft (NL)

(21) Appl. No.: 15/282,845

(22) Filed: Sep. 30, 2016

Related U.S. Application Data
(63) Continuation of application No. PCT/NL2015/050200, filed on Mar. 30, 2015.

(30) Foreign Application Priority Data
Mar. 31, 2014 (NL) 2012534

(51) Int. Cl. G01N 21/552; G01N 33/28; G01N 33/00

(52) U.S. Cl. CPC G01N 21/553; G01N 33/005; G01N 33/0016; G01N 33/2841

(57) ABSTRACT

A single element thin-film device, a method for producing a thin-film device, a single element for detecting hydrogen absorption, a hydrogen sensor, and an apparatus for detecting hydrogen and to an electro-magnetic transformer comprising such sensor. A thin-film device comprises a substrate, an active sensing layer whose optical properties change depending on hydrogen content, and a protective layer on the active sensing layer.

### CROSS-REFERENCE TO RELATED APPLICATIONS

This application is a continuation application of Patent Cooperation Treaty Application No. PCT/NL2015/050200, filed on Mar. 30, 2015, which claims priority to Netherlands Patent Application No. 2012534, filed on Mar. 31, 2014, and the specifications and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND OF THE INVENTION

Field of the Invention (Technical Field)

The present invention relates to a single element thin-film device, to a method for producing a thin-film device, to a single element for detecting hydrogen absorption, to a hydrogen sensor, to an apparatus for detecting hydrogen and to an electro-magnetic transformer comprising said sensor.

Description of Related Art including information disclosed under 37 C.F.R. §§1.97 and 1.98

In a more generic perspective in an economy with hydrogen as a major energy carrier, the development of affordable, reliable, sensitive and selective hydrogen sensors is indispensable. Several types of hydrogen sensors are currently available, which exploit the following detection mechanisms: catalytic, electrochemical, mechanical, optical, acoustic, thermal conductivity, resistance and work function. In principle, Pd-based optical fiber sensors could meet requirements if cross-contamination effect of a Pd surface by oxygen, moisture or carbon monoxide, for example, can be prevented. Such sensors can also be used for detecting hydrogen in other environments. Since hydrogen detection often takes place in an explosive environment, cf. for leak detection or hydrogen-concentration measurements in gas streams, use of optical hydrogen sensors has a major advantage of being intrinsically safe due to the lack of electric leads in a sensing area. In addition known, fiber-optic, Pd-based thin-film hydrogen sensors represent a relatively cheap and reliable solution to this problem since they also allow for continuous sensing via remote hydrogen-gas detection, a key for personal and material safety. However, it is well known that Pd-based sensors have a highly non-linear optical response, depending strongly on the applied hydrogen pressure.

A prior art thin-film device comprises a substrate, an active sensing layer whose optical properties change depending on hydrogen content, and having a protective layer on the active sensing layer.

Such thin-film devices are known from the prior art. As an example, WO2007/126313 discloses a switchable mirror device comprising an active layer, wherein said active layer changes its optical properties upon addition or removal of hydrogen and comprises a hydrogen and oxygen permeable and water impermeable layer, wherein said layer is liquid water impermeable and water vapor permeable and has hydrophobic surface properties.

In a further example, WO2007/049965 A1, an optical switching device is recited. In such a device auxiliary layers may be present, such as for protection. Such layers typically are rather thick. The layers disclosed therein relate solely to Mg alloys, i.e. always comprising Mg and a further metallic element.

Recent improvements to extend a range of measurement, and at the same time retaining sufficient optical contrast, relate to sensor materials comprising alloys, typically of at least two elements. Such sensors and alloys often suffer from hysteresis. On top of that, for these alloys a range of hydrogen detection is still relatively small (maximum 4 orders of magnitude) and a minimum detectable concentration (detection limit) (at a given temperature) is relatively high.

Incidentally some prior art documents relate to materials for hydrogen detection. The materials used typically relate to alloys or oxides. The oxides may be reduces partly at the most to metals; hence the material will always comprise oxygen and most likely also water.

For example, WO 2003/048753 A2 recites a method and a sensing element for measuring the flow density of atomic hydrogen. An electrically conductive thin-film of the sensing element is exposed to the flow of atomic hydrogen during a certain exposure time, and a time variation of the electrical resistance of the thin film of the sensing element is measured during a time period within the exposure time. The measured time variation is utilized to determine the flow density of atomic hydrogen.

This document is totally irrelevant for the present invention, as use is made of electrical resistance, which requires electrical components, being inherently very dangerous, as mentioned above. Also a catalyst layer is not present. Further, the Ti layers therein do not relate to detecting layers, but to adhesion layers.

Mak et al. in Sensors and Actuators B: Chemical, Vol. 190, (2014-01-01), p. 982-989 recites an optical fiber sensor for continuous monitoring of hydrogen in oil. The optical detecting layer therein comprises a MgTi alloy.

Hosoki et al. in Sensors and Actuators B: Chemical, Vol. 185, p. 53-58 recites a surface plasmon resonance hydrogen sensor using Au/Ta₂O₅/Pd multi layers on hetero core optical fiber structures. Clearly the oxide is only used as a spacer layer.

Perroton et al. in Optics Express, Vol. 19, No. S6, Nov. 7, 2011 (p. A1175) recites a fiber optic Surface Plasmon Resonance sensor based on wavelength modulation based on an optical change in Pd due to hydrogen absorption, for hydrogen sensing.

US2007/089989 A1 recites a hydrogen gas leak detector comprises a thin film hydrogen detector on a sheet of conformable substrate material, for example, a plastic cling wrap material or a plastic heat shrink material, that is wrappable around a component from which hydrogen gas might leak or evolve. The thin film hydrogen detector may comprise a thin film hydrogen detecting material, for example, a metal oxide, and a thin film catalyst material. The conformable substrate material can be transparent or translucent. As an example of the metal oxide vanadium oxide is mentioned.

JP S61 204545 A seems to recite an optical detecting layer having TiO₂.

The present invention therefore relates to a thin-film device and further aspects thereof, which overcomes one or more of the above disadvantages, without compromising functionality and advantages.

### BRIEF SUMMARY OF THE INVENTION

It is an object of the invention to overcome one or more limitations of the thin-film devices of the prior art and at the very least to provide an alternative thereto.

In a first aspect, the invention relates to a thin-film device according to claim 1, consisting of a single transition metal. It is noted that in use the single transition metal layer may comprise hydrogen absorbed therein, typically absorbed interstitially. It is noted that none of the above prior art documents relate to optical sensor consisting of layers of a single transition metal. It is noted that it is very unexpected that pure metal layers would have optical properties which could be used for, e.g., hydrogen detection.

With respect to the term “single” a relatively pure metal is indicated, having typically only very low amounts of impurities incorporated therein. Part of these impurities is, e.g., absorbed due to natural occurring processes. Typically a total amount of impurities, such as oxygen and other metals, is less than 5 atom % (based on the total of atoms) and preferably less than 2 atom %.

The present invention relates to a new class of optical hydrogen sensing materials, consisting of a thin film of a single material. The present invention came as a surprise to inventors; in literature, there are no reports of the optical response as function of the hydrogen concentration (or partial hydrogen pressure) for Group 4 (Ti, Zr, and HO and Group 5 elements (V, Nb, and Ta). Transmission, reflection and/or absorption of light by the present layers changes through addition or removal of hydrogen from the layer. Such changes can be measured. It is noted that in a (prior art) alternative Pd may be used for sensing hydrogen; however, the solubility of hydrogen in the alpha and beta phase thereof changes hardly as a function of pressure. As a result only a very small optical signal can be obtained in the Pd layer.

Unexpectedly, the present thin film provides over a large range of hydrogen concentrations (at least 7 orders of magnitude) and at low hydrogen concentrations (levels as low as a few ppb at 90° C. and 120° C.) a one-to-one optical response in at least the visible/near-infrared part of the spectrum. The large range may further provide the advantage of requiring only one (or two) sensors to monitor a hydrogen pressure, instead of a range of sensors. It was found that this response is over the whole range of hydrogen concentrations the same for both the absorption and desorption (no hysteresis). The optical contrast may be somewhat low, but is still considered sufficient. Inventors performed detailed experiments on two metals (Hafnium and Tantalum), and found a similar effect in other transition metals, such as Titanium, Zirconium, Vanadium, and Niobium. A further advantage is that segregation typically being an optional failure mode in multiple elements hydrogen sensors is avoided. In this respect it is observed that there is a principle difference between a detector and a sensor. The detector is only capable of detecting presence of a physical/chemical entity, whereas the sensor is capable of determining a quantity of the physical/chemical entity, in other words the sensor responds to a stimulus of the physical/chemical entity and provides a result in the form of another (variable) physical/chemical entity that is representative for the quantity. For a sensor it is important that for instance an optical contrast obtained as a result of changing concentration of, e.g., hydrogen is built up gradually in order to precisely determine the concentration.

It is noted that the present invention provides a controlled and reliable absorption, specifically of hydrogen, over a large range of hydrogen pressures, without hysteresis. The present device therefore provides a well-defined relation between a hydrogen concentration and an optical response (in the optical sensing layer). As the mechanism of absorption is in principle reversible, also controlled and reliable desorption is provided. As such the present device is capable of monitoring fluctuations in hydrogen concentration; in case of a device with hysteresis such is very complicated, or impossible.

In an example a means for monitoring a (varying) hydrogen concentration over a large range of pressures is provided. It is noted that the present optical system is much safer to use and to handle compared to, e.g., electrical (conducting) sensors, especially in environments where a large electro-magnetic field may be present.

By using on single element also a more stable and robust device is provided, compared to prior art (alloy) devices.

In an example of an application of the present thin film detection and measurement of low concentrations hydrogen produced by slow processes where a continuous detection is necessary, is considered. The absent of a hysteresis makes it possible to use the present thin film especially for processes where the hydrogen concentration fluctuates.

A further application relates to the detection of hydrogen in power transformers by means of optical fibers, where the concentration of hydrogen (in oil) is considered indicative for aging of the insulation oil.

Another application relates to detection of (small) leaks. Hydrogen gas—as the smallest molecule—can be used to test presence of small leaks. By means of optical fibers, the present sensing material is used to detect small leaks. Such detection can take place over a long period of time, and in small areas which are difficult to reach.

In an example the present thin-film device comprises a substrate, an active sensing layer whose optical properties change continuously as a function of hydrogen content, a Pd cap layer to dissociate hydrogen which acts simultaneously as a protective coating for the sensing layer, and a protective layer coating the Pd which protects this cap layer. A protective layer clearly is something different than e.g. a sensing layer. In the following sections various elements of the present device are further elucidated.

A person of skill in the art is able to identify many suitable substrate materials upon which a thin-film device such as the thin-film device of the invention can be constructed. Examples of suitable substrate materials include glass, quartz, indium-tin oxide, etc. The substrate material is preferably optically transparent (more than 95%), at least over a proportion of the visible, UV and/or IR regions of the electromagnetic spectrum (200 nm-3000 nm). Such provides for use of white light, IR-light, UV-light, a laser with a specific wavelength, and combinations thereof.

As mentioned above, optical sensing layers, with variable optical properties depending on e.g. a hydrogen content of the layer, e.g., comprising an alloy, are known in the prior art. In an example of the present invention the sensing layer consist of the present single transition metal.

The optical sensing layer may be in a sequence of layers or layer stacks or in 2- or 3-dimensional domains.

A catalyst such as in a layer is provided on top of the optical sensing layer, such as coating the optical sensing layer. Examples of such layers include for example Pd-layers. The Pd-layers may comprise pure Pd or mixtures comprising Pd. For example, Ag can be added in a quantity of for example 20-30 mole %. The catalyst may also relate to a complex layer, suited for the present purpose. Such layers serve to facilitate hydrogen absorption by the optical sensing layer.

It is noted that a term as “on top” may relate to a sequence of, e.g., layers, a first layer coating a second layer, a layer provided on an intermediate layer, the intermediate pro-vided on, e.g., the sensing layer, etc. The layer may also partly be on top. In view of the present application such terminology is mainly functional of nature.

On top of the optical sensing layer, or on top of the catalyst (layer), a protective layer is provided, the protective layer not limiting functionality of the optical sensing layer, e.g., being permeable to relevant species, and protecting the optical layer. Both the catalyst layer and the protecting layer are permeable to a species to be measured, such as hydrogen, and are optically transparent, at least over a range of the visible, UV and/or IR regions of the electromagnetic spectrum. An example of a protecting layer from the aforementioned WO2007/126313 is to provide a layer of Teflon™. The protective layer is provided to improve the longevity of the thin-film device through preventing deterioration of the catalyst and/or optical sensing layers and improves the handleability of the device through pre-venting a user from coming into contact with the optical sensing and/or catalyst layers. It is noted that the nature of Teflon™ and more specific sputtered PTFE makes it in principle difficult to process.

Control and reliability of, e.g., hydrogen absorption is further achieved with the thin-film device of the invention by providing an optical sensing layer according to the invention.

Examples of coating layers are given in the Dutch Patent Application NL2010031, filed Dec. 20, 2012. Details, teachings and examples thereof are incorporated by reference.

The present single transition metals provide in an example for a range of hydrogen pressures between 1*10⁻⁴ Pa-1*10³ Pa (at elevated temperatures (90° C. and 120° C.)) to be detected accurately. Depending on the present metal much lower pressures (e.g., 10⁻⁵ Pa) and much higher pressures (e.g., 10⁷ Pa) may be detected. In comparison an optimal crystalline MgTi layer provides 1-2 orders of hydrogen pressure (˜1*10² Pa-˜1*10⁴ Pa at 120° C.) to be detected accurately.

Desirable performance of the thin-film device of the invention in terms of control and reliability of hydrogen absorption can be achieved through either improvement separately or through the combination of improvements. Reliability relates particularly to reliability over time, such as tens of years, and with repeated use.

The invention also relates to a hydrogen sensor and to an electro-magnetic transformer comprising said hydrogen sensor.

The present invention provides a solution to one or more of the above mentioned problems and overcomes drawbacks of the prior art.

Advantages of the present description are detailed throughout the description.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 shows applied hydrogen pressure (bottom) and measured transmittance (top) as function of time.

FIG. 2 shows applied hydrogen pressure (bottom) and measured transmittance (top) as function of time.

FIG. 3 shows applied hydrogen pressure as a function of measured transmittance at 120° C. (top) and 90° C. (bottom).

FIG. 4 shows results of a measurement of applied hydrogen pressure (bottom) and measured reflectance (top) as function of time.

FIG. 5 shows results of a measurement of applied hydrogen pressure (bottom) and measured transmittance (top, Hf and Ta respectively)) as function of time.

### DETAILED DESCRIPTION OF THE INVENTION

In an exemplary embodiment, the catalyst layer has a thickness in the range of 1.5-500 nm, preferably 3-100 nm, such as 5-30 nm.

In an example the present device further comprises one or more intermediate layers, wherein the intermediate layer preferably comprises a Period 4 transition metal, such as Ti, even more preferably an alloy of (i) a Period 4 transition metal, such as Ti, and (ii) the single transition metal or a second metal. For instance, when the present single element (of the optical sensing layer) is Ti, the intermediate layer may comprise TiZr.

It may be preferred to provide two intermediate layers, one between the catalyst and present alloy, and one between the present alloy and substrate.

In an exemplary embodiment, the intermediate layer has a thickness in the range of 1.5-500 nm, preferably 3-100 nm, such as 5-30 nm.

In an exemplary embodiment the optical sensing layer has a thickness in the range of 1.5-500 nm, preferably 10-100 nm, more preferably 20-50 nm.

In an exemplary embodiment the protective layer has a thickness in the range of 0.02-200 μm.

In an exemplary embodiment the protective layer and the catalyst layer are combined, e.g., are one and the same.

Also a catalyst layer to enhance hydrogen absorption is present, typically on top of the optical layer, either directly or with one or more intermediate layers.

In an exemplary embodiment in use the transition metal comprises hydrogen in an amount of [transition metal (TM)]:[H] of [1,0.5] (equal amounts to twice as much hydrogen), preferably [0.75,0.5]. That is, the transition metal, such as Hf, is partly filled with hydrogen when exposed to hydrogen; as such the technical nature of the optical sensing layer changes as hydrogen is incorporated therein. The hydrogen, even at very low (external) pressures, remains partly in the transition metal. Variation in hydrogen pressure is found to result in a filling of the transition metal with hydrogen in a range of approximately TMH₁ to TMH₂, preferably TMH_{1.5} to TMH₂, such as TMH_{1.6} to TMH₂.

The present embodiments may be combined. An example is wherein the optical sensing layer comprises at least two layers, each layer consisting of a different transition metal. Therewith for instance a larger hydrogen pressure sensing range may be obtained. In an example thereof a layer of Hf is combined with a subsequent layer of Ta. Other combinations are (layers of) Hf:Ti, Hf:Zr, Hf:V, Hf:Nb, Ta:Ti, Ta:Zr, Ta:V, Ta:Nb, Ti:Zr, Ti:V, Ti:Nb, Zr:V, Zr:Nb, and V:Nb. Likewise also three layers may be considered. Each individual layer may have a thickness of 0.2 nm-100 nm, preferably 1 nm-500 nm, more preferably 2 nm-250 nm, such as 5 nm-100 nm.

A further example of a combination is wherein the optical sensing layer comprises at least two domains, each domain consisting of a different transition metal layer. Combinations as above are considered. Further also a combination of layers and domains is considered. Herewith a large degree of freedom in design and performance is obtained.

In an exemplary embodiment the domain has a size of 10⁻⁵-10⁹ μm², such as having a width of 0.01-5*10⁴ μm and a length of 0.01-5*10⁴ μm. The domain(s) may be rectangular, such as square, hexagonal, polygonal, circular, and combinations thereof.

In an exemplary embodiment the device is for use in a frequency range of 200-3000 nm. In other words, the present device can be used over a broad range of frequencies. If applicable, e.g., in terms of further optimization, one frequency may be used, and likewise at least one frequency band having a certain width. Therewith a more sensitive device can be obtained.

In a second aspect, the invention relates to a method of producing the present optical thin-film device comprising providing a substrate, depositing an optical sensing layer on the substrate, the optical sensing layer consisting of a single transition metal, the metal being selected from Hf, Ta, Ti, Zr, V and Nb, preferably from Hf and Ta. It may further comprise depositing a catalyst layer on the optical sensing layer, and providing a protective layer on the catalyst layer.

It has been found experimentally that for a stable performance a device is first cycled a few times, from a relatively low (hydrogen) pressure to a relatively high (hydrogen) pressure, and back. 2-10 cycles are typically sufficient, such as 3-5 times. It is also preferred to cycle at elevated temperature; fewer cycles are required in that case, compared to ambient temperature cycling.

In a third aspect, the invention relates to a use of a layer consisting of a single transition metal, the metal being selected from Hf, Ta, Ti, Zr, V and Nb, preferably from Hf and Ta, for optically detecting a chemical species, such as hydro-gen, especially for detecting low concentrations. As mentioned above, it has come as a surprise that a single transition metal can be used for detecting hydrogen over a wide pressure range.

In an example of said use a change in optical properties is used to detect a change in concentration of the chemical species, such as hydrogen.

In a further example of said use a change in electrical properties is used to detect a change in concentration of the chemical species, such as hydrogen. A change may be detected in a layer of the present single transition metal, in a layer comprising conglomerates of (nano)particles of the single transition metal, and combinations thereof. As such the present invention also relates to a layer, a conglomerate layer, and combinations thereof, consisting of the present single transition element, applied in the present thin film device and sensor, respectively. With respect to the nanoparticle these may be present in a layer like structure, each individual nanoparticle having a surface area contributing to the surface area of the present layer of 4 nm²-1 μm²; in this respect the present layer may be considered as a grain-like structure.

It is noted that various methods of the prior art are not reliable, not accurate, expensive, and often not applicable at all, especially in complex and/or harsh environments. Specifically the present invention provides for detection of species, e.g., hydrogen gas species, in oil, such as transformer oil. It is noted that the species are an indirect measurement for the quality and/or status of the transformer as a whole and of sub-functionality thereof, such as transformer oil. As a consequence the quality and status of the transformer can now be monitored continuously.

In a fourth aspect, the invention relates to an optical sensor comprising the thin-film device of the invention. In a preferred example the sensor is a hydrogen sensor. The sensor may be provided with an optical transmitter, such as an optical fiber. Such provides e.g. as advantage that a measurement can take place at a spatial distance of detection. Even further the invention may relate to a combination of optical sensing layers, such as a stack of layers. Each layer or stack of layers may be optimized to sense a species, such as hydrogen, oxygen, nitrogen, carbon monoxide, carbon dioxide, etc. Also, a layer or stack of layers may be optimized to determine a species in a first concentration range, and a further layer or stack of layers for determining a species in a second concentration range. Likewise and preferred a combination of various 2-D and 3-D domains may be used. Thereby an enlarged range of concentrations can be determined. Even further the sensor may comprise one or more of the above, e.g., layers for various species and layers for various concentrations of one or more species. Even further, other materials may be used in combination with the present optical layer to extend, e.g., a pressure range and to incorporate further species being measurable. An advantage is that the present invention allows for a combination of various optical layers without much extra measures to be taken in order to obtain a functional device.

In a fifth aspect, the invention relates to an electro-magnetic transformer comprising the optical hydrogen sensor of the invention. Therewith behavior and status of the transformer can be monitored. Even further, an automatic signal may be provided, indicating malfunction or risk of malfunction, based on the hydrogen concentration measurement. The transformer can then be replaced or serviced, as required.

In an sixth aspect the invention relates to an apparatus for optically detecting hydrogen comprising a sensor, the sensor being located at a longitudinal side of an optical transmitter, the optical transmitter comprising a central transmitting element, such as a quartz core, a transducer layer, preferably having a surface plasmon resonance frequency, the present single transition element according to the invention, and optionally a protection layer, preferably according to the invention, and a frequency shift detector. It is noted that the (geometrically) configuration of the present apparatus is slightly different form the present sensor above.

With the optical resonator in combination with the frequency shift detector a resolution in the order of pm is obtained.

The above apparatus relates to a new design of a fiber optic Surface Plasmon Resonance (SPR) sensor using the present single transition element according to the present invention. In an example, a transducer layer is deposited on the outside of a multimode fiber, after removing the optical cladding thereof. In an example the transducer layer is a multilayer stack made of silver, silica and the sensing layer (e.g., the single transition metal element, a Pd-alloy and the protective coating). Spectral modulation of light transmitted by the fiber allows detecting the presence of hydrogen in the environment. The sensor is only sensitive to a Transverse Magnetic polarized light and Traverse Electric polarized light can be used therefore as a reference signal. A more reliable response is expected for the fiber SPR hydrogen sensor based on spectral modulation instead of on intensity modulation. The multilayer thickness defines the sensor performance. The silica thickness tunes the resonant wavelength, whereas the Silver and Palladium thickness determine the sensor sensitivity. In a comparative configuration (NA=0.22, 100 pm core radius and transducer length=1 cm), a resonant wavelength is shifted over 17.6 nm at a concentration of 4% Hydrogen in Argon for the case of the 35 nm Silver/100 nm Silica/3 nm palladium multilayer. Amongst others the above comparative results are published in two articles of one of the present inventors (Opt. Soc. America, 7 Nov. 2011, Vol. 19, No. S6, ppA1175-1183 and Proc. SPIE, Vol. 8368, pp. 836804-1-12).

The invention will hereafter be further elucidated through the following examples which are exemplary and explanatory of nature and are not intended to be considered limiting of the invention. To the person skilled in the art it may be clear that many variants, being obvious or not, may be conceivable falling within the scope of protection, defined by the present claims.

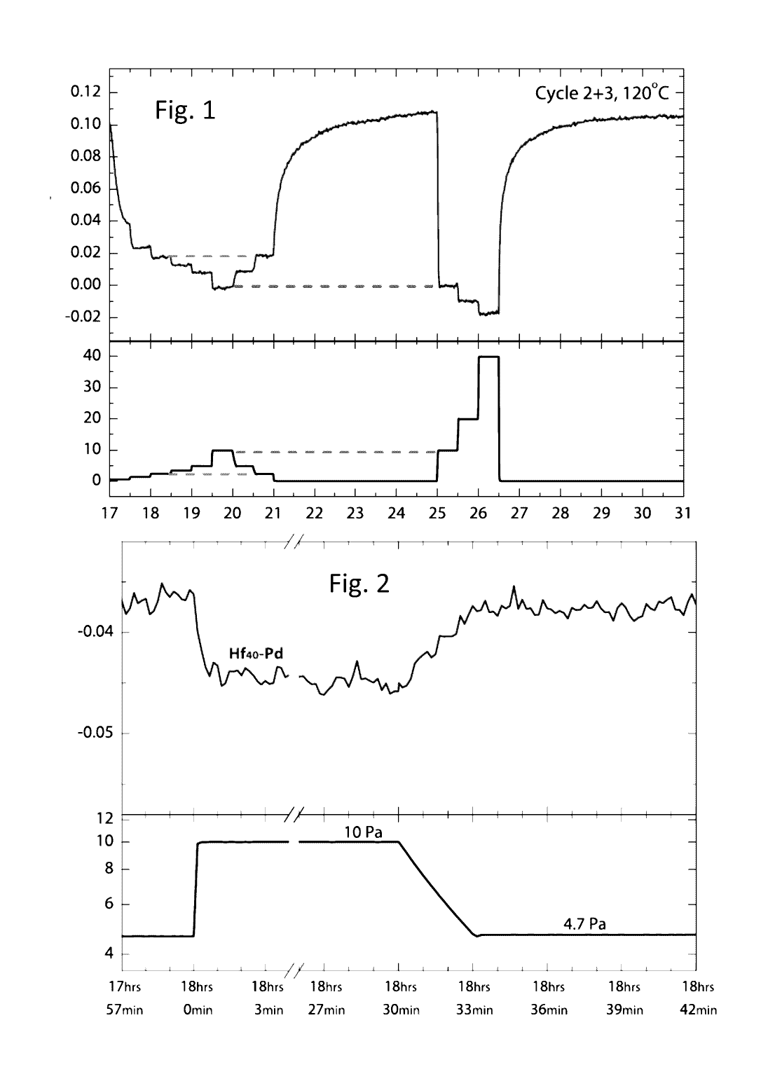

FIG. 1 is a measurement of the applied hydrogen pressure [Pa]and the transmittance In(T/T0) of a 40 nm Hf layer capped by a 10 nm Pd layer as function of time (cycles 2+3)[hours]. It shows that, at elevated temperature, the same optical transmittance is obtained when exposing the device to the same pressure, independent of the history (increasing pressure, decreasing pressure, or after cycling) of hydrogen exposure. This figure indicates the absence of hysteresis and short-term stability.

FIG. 2 is again a measurement of the applied hydrogen pressure [Pa] and the transmittance In(T/T0) of a 40 nm Hf layer as function of time [hours], but now for a shorter time period. This figure indicates that, at elevated temperature, the response of the optical transmittance is one to one with the applied hydrogen pressure, independent of increasing or decreasing the pressure. This figure indicates the very fast response of Hf to a small increase/decrease of the hydrogen pressure.

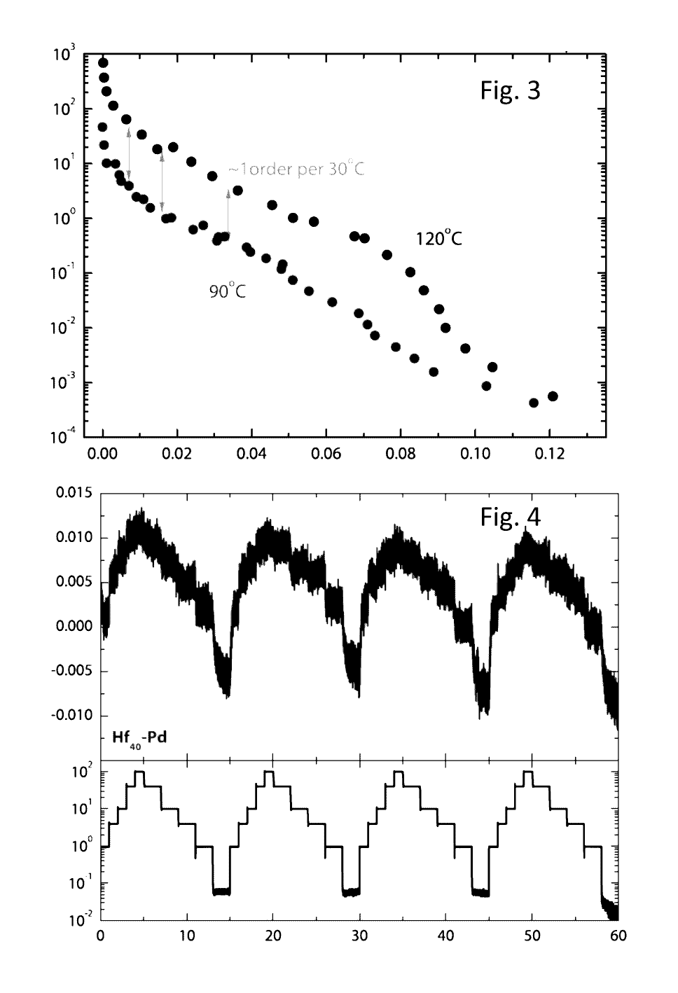

FIG. 3 is a measurement of the applied hydrogen pressure [Pa] as function of the transmittance for a 40 nm Hf layer at two elevated temperatures (90° C. and 120° C.). It shows that at 120° C. there is a well-defined relation between the hydrogen pressure and the optical transmittance for at least 7 orders of magnitude. It also shows that decreasing the temperature to 90° C. results in a (relative to 120° C.) decrease of the pressure range with approximately one order of magnitude.

FIG. 4 is a measurement of the applied hydrogen pressure [Pa] and the optical reflectance In(R/R0) as function of time (for 4 cycles). This figure shows the hysteresis-free steps observed in transmittance also are observed in reflectance. This result is considered essential for application of a Hf sensing-layer in an optical fiber-sensor.

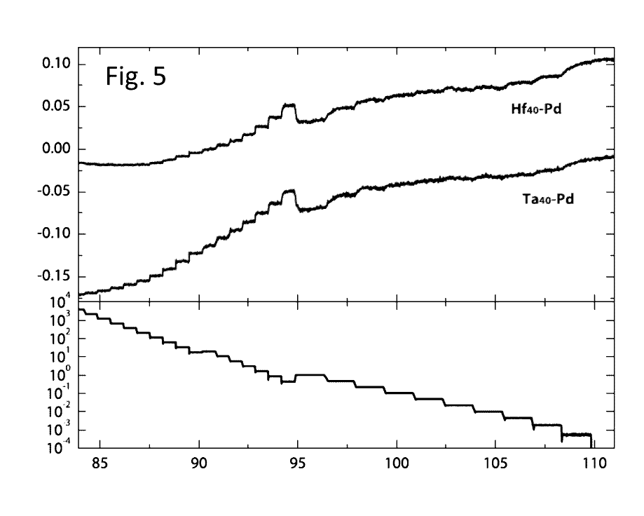

FIG. 5 is a measurement of the applied hydrogen pressure [Pa] and the transmittance In(T/T0) of a 40 nm Hf layer and a 40 nm Ta layer versus time [hours]. It shows that Ta, at elevated temperature, shows an almost identical behavior as Hf. In fact, Ta shows more distinct steps at higher pressures compared to Hf.

### EXAMPLES

### Experimental

### Preparation

Thin films of Hafnium and Tantalum, respectively, with a thickness of 40 nm are deposited on a quartz substrate by means of DC magnetron sputtering. To promote the hydrogen dissociation and to prevent the sensing layer from oxidation, the sensing layer is covered with a Pd-layer (10 nm).

### Characteristics

### a) Range of Hydrogen Detection

At 120° C., for Hafnium inventors observed an optical response between 10⁻⁴ and 200 Pa; for Tantalum inventors observed an optical response between 10⁻⁴ and 103 Pa. After further improvement, a larger range than the previous seven orders of magnitude is to be found. It is noted that a pressure of 10⁻⁴ Pa is the lower limit of inventors equipment and no saturation of the optical contrast is obtained at pressures close to this lower limit. Thus, at least at a lower pressure (smaller than 10⁻⁴ Pa) is to be expected.

It is observed that at 90° C. the above range shifts down with approximately one order of magnitude.

### b) Hysteresis

Inventors found no indication of hysteresis as in both the absorption and desorption of hydrogen a same level of hydrogen pressure results in a same level of optical contrast.

### c) Optical Contrast & Resolution

The optical contrast is low compared to, e.g., Mg-based sensing materials, but comparable to the optical contrast of Pd-based materials. Despite the relative low optical contrast, inventors are able to obtain—in reflection, with a primitive setup—a resolution of less than half an order of magnitude of hydrogen concentrations. Such is considered sufficient and can be improved further.

### d) Response Time

The response time of the present material is found to depend on the hydrogen concentration. At 120° C., at high concentrations (>10⁻¹ Pa) the optical response is found to relate one-to-one compared with the response of the hydrogen concentration. However, at low concentrations (<10⁻¹ Pa) the response time (in desorption) of the optical contrast is six times larger than the response time of the hydrogen concentration. Optical measurements shows that a 40 nm thick Hafnium film shows the best optical contrast/response time ratio.

### e) Stability

The configuration used shows a good stability. Even after more than twenty hydrogenation cycles, there is a good optical response. However, due to instability of the light source inventors are not yet able to conclude definitely if there is actually a degradation in optical contrast. For Hafnium inventors observe a clear optical response to different hydrogen pressures, even after exposure of the film for more than one month to (open) air.

### f) Considerations

The present experiments indicate that the Group 4 elements show hysteresis free behavior of optical response between hydrogen concentrations of (in case of Hf) HfH_{1.63} and HfH₂. It has been found that HfH_{1.63} has an FCC structure, whereas HfH₂ shows an FCT structure. It is considered that this structure change is also present in TiHₓ and ZrHₓ. It is also considered that the FCC-FCT structure change causes the observed optical response. It was found that the Group 5 elements show a BCC-BCT transition, which is considered very similar to the FCC-FCT transition of Group 4 elements. Therefore it is considered that the optical response as function of the hydrogen pressure in Group 5 elements has the same origin as in Group 4 elements.

Although the invention has been described in detail with particular reference to these embodiments, other embodiments can achieve the same results. Variations and modifications of the present invention will be obvious to those skilled in the art and it is intended to cover in the appended claims all such modifications and equivalents.

What is claimed is:

1. An optical measuring device allowing controlled and reliable optical measurement of large range molecular hydrogen pressure comprising:
(a) a substrate;
(b) at least one optical thin film sensing layer of 5-1000 nm thickness on the substrate, the at least one optical sensing layer consisting of a single transition metal, the metal being selected from the group consisting of Hf, Ta, Ti, Zr, V and Nb, the optical sensing layer having optical properties that may change continuously as a function of hydrogen content;
(c) a protective layer with a thickness in the range of 0.02-200 pm provided on the optical sensing layer either directly or through an adhesive layer;
(d) a catalyst layer with a thickness in the range of 1.5-500 nm between the optical sensing layer and the protective layer; and
(e) an optical sensor for measuring optical properties in a wavelength range of 200-3000 nm of the optical sensing layer.

2. The thin-film device according to claim 1, wherein the optical sensing layer has a thickness in the range of 1.5-500 nm, and wherein the protective layer has a thickness in the range of 0.02-200 μm.

3. The thin-film device according to claim 1, wherein the protective layer and the catalyst layer are combined.

4. The thin-film device according to claim 1, wherein the transition metal is capable of comprising hydrogen in an amount of [transition metal (TM)]:[H] of [1,2].

5. The thin-film device according to claim 1, wherein the optical sensing layer comprises at least two layers, each layer consisting of a different transition metal.

6. The thin-film device according to claim 1, wherein at least one optical sensing layer comprises at least two domains, each domain consisting of a different transition metal.

7. The thin-film device according to claim 6, wherein the domain has a size of 0.01-10⁸ μm².

8. A method for producing a thin-film device according to claim 1, comprising:
providing a substrate, depositing an optical sensing layer on the substrate, the optical sensing layer consisting of a single transition metal, the metal being selected from the group consisting of Hf, Ta, Ti, Zr, V and Nb;
providing a catalyst layer;
providing a protective layer; and
cycling the device 1-10 times from a relatively low (hydrogen) pressure to a relatively high (hydrogen) pressure, and back.

9. Use of a layer consisting of a single transition metal, the metal being selected from the group consisting of Hf, Ta, Ti, Zr, V and Nb, in a device according to claim 1 for optically detecting a chemical species.

10. A sensor comprising at least one device of claim 1, further comprising an optical transmitter, wherein the optical sensing layer is located at a top of the optical transmitter and wherein the optical sensing layer is located at a longitudinal side of the optical transmitter.

11. An electro-magnetic transformer comprising an optical hydrogen sensor according to claim 10.

12. An apparatus for optically detecting hydrogen comprising a sensor according to claim 10 wherein the sensor is located at a longitudinal side of an optical transmitter, the optical transmitter comprising:
a central transmitting element;
a transducer layer, a layer consisting of a single transition metal for detecting hydrogen, the metal being selected from the group consisting of Hf, Ta, Ti, Zr, V and Nb; and
a frequency shift detector.
